(19) United States
(12) Patent Application Publication
(10) Pub. No.: US 2006/0141638 A1
(43) Pub. Date: Jun. 29, 2006

(54) Equine relaxin as a measure of treatment efficacy

(76) Inventors: Peter L. Ryan, Starkville, MS (US); Carol Bagnell, North Brunswick, NJ (US)

Correspondence Address: LICATLA & TYRRELL P.C., 66 E. MAIN STREET, MARLTON, NJ, 08053, US

(21) Appl. No.: 11/356,602

(22) Filed: Feb. 17, 2006

Related U.S. Application Data
(63) Continuation of application No. 10/079,040, filed on Feb. 20, 2002.
(60) Provisional application No. 60/270,971, filed on Feb. 22, 2001.

(51) Int. Cl. G01N 33/53

(52) U.S. Cl. 436/510

(57) ABSTRACT

A method for predicting treatment efficacy in pregnant mares affected by a disease or condition that alters placental function and results in a problematic pregnancy or delivery in the mare that is related to measurement of plasma relaxin levels is provided.

This application is a continuation of U.S. Ser. No. 10/079,040 filed Feb. 20, 2002 which claims the benefit of priority from U.S. provisional application Ser. No. 60/270,971, filed Feb. 22, 2001.

### BACKGROUND OF THE INVENTION

Pregnant mares with at risk pregnancies can be assigned to three categories which include: mares with a history of problematic pregnancies, deliveries and/or birth of compromised foals; mares with reproductive abnormalities, premature udder development or systemic illness; and mares with no apparent risk factors that still experience an abnormal periparturient event. Examples of recurrent periparturient events include premature placental separation, dystocia, placentitis, premature delivery, and prolonged gestation. Late term fetal and perinatal death represents a major emotional and financial loss for horse breeders. A review of case records of 3500 aborted fetuses, still-born foals, and foals that died within 24 hours of birth revealed that over 60% of the cases were associated with some form of placental insufficiency such as placentitis, premature placental separation, or placental thickening (Giles, R. C. et al. 1993. J. Am. Veter. Med. Assoc. 203:1170-1175). Subsequent studies have shown that placentitis is the single most common factor contributing to late term abortion and perinatal death in foals (Hong, C. B. et al. 1993. J. Veter. Diag. Invest. 5:550-566). Untreated placentitis can lead to premature delivery, neonatal sepsis and peripartum hypoxia, which leads to either mortality or survival with expensive intensive care.

Another factor that contributes to placental insufficiency is fescue toxicosis resulting from grazing of endophyte-infected tall fescue. Tall fescue, which is an important forage grass for horses in the United States, is often infected with the endophyte Acremonium coenophialum, an organism that produces ergot alkaloids. Consequences of the ergot poisoning include placental thickening, premature placental separation, complicated deliveries, prolonged gestation, dystocia, agalactia, and high foal mortality (Cross, D. L. et al. 1995. J. Anim. Sci. 73:899-908).

With the high expense of horse breeding and the long gestation period of a mare, early and reliable identification of placental insufficiency are desirable. If diagnosed early, placentitis can be treated with systemic antibiotics, progestins, non-steroidal anti-inflammatory drugs, and/or steroids. Likewise, early detection of fescue toxicosis in pregnant mares could allow for timely medical and/or dietary intervention to rescue the pregnancy. Therefore, a biomarker that reflects placental and fetal well-being in the horse is desired. An easily detectable biomarker, such as an hormonal marker, would be useful as a way to monitor both the early development of placental insufficiency as well as a way to monitor efficacy of treatments for placental insufficiency.

Research has focused on evaluation of equine placental function in a non-invasive manner. Serum-based hormone assays have been used to determine pregnancy and fetal viability in the horse (Terqui, M. and E. Palmer. 1979. J. Reprod. Fertil. 27:441-446; Stabenfeldt, G. H. et al. 1991. J. Reproduct. Fertil. Suppl. 44:37-44). However, available assays have not been useful in predicting problematic pregnancies in late gestation. There is currently no simple hormonal diagnostic assay available for use by veterinarians to predict problematic pregnancies and deliveries, which would result in a reduction in the incidence of late-term abortions and perinatal deaths in horses.

### SUMMARY OF THE INVENTION

The present invention is a method for predicting treatment efficacy in pregnant mares affected by a disease or condition that alters placental function and results in a problematic pregnancy or delivery in the mare which comprises measuring the levels of relaxin in plasma of a pregnant mare before administration of a drug or treatment, administering the drug or treatment to the mare, and measuring the levels of relaxin in plasma of the mare following administration of the drug or treatment from the first day of drug or treatment administration until the time of delivery in the mare, wherein a failure of the plasma relaxin levels to increase following drug or treatment administration is indicative of a problematic pregnancy or delivery in the mare.

Another object of the present invention is a diagnostic kit for predicting treatment efficacy in pregnant mares affected by a disease or condition that alters placental function and results in a problematic pregnancy or delivery in the mare which comprises a means for detecting levels of relaxin in plasma of a pregnant mare before administration of a drug or treatment, and a means for detecting levels of relaxin in plasma of the mare following administration of the drug or treatment from the first day of drug or treatment administration until the time of delivery in the mare.

### DESCRIPTION OF THE DRAWINGS

FIG. 1 illustrates the differences in levels of circulating relaxin levels in gravid pony mares grazed on endophyte-infected pasture that experienced either normal or problematic pregnancies that were attributed to fescue toxicosis. Plasma relaxin levels were determined by radioimmunoassay and were consistently lower in mares that experienced complications of pregnancy (p=0.03).

FIG. 2 illustrates the differences in systemic relaxin levels in individual mares that were treated with either saline (untreated) or fluphenazine (treated) after grazing on endophyte-infected fescue pasture. Plasma relaxin levels were depressed in the two mares that exhibited signs of complicated pregnancies consistent with fescue toxicosis.

FIG. 3 illustrates systemic relaxin levels in two individual mares treated with fluphenazine that had different pregnancy outcomes. Mares were grazed on endophyte-infected fescue and then both treated with fluphenazine. However, in the mare that did not respond to treatment (exhibited a pregnancy complication) the plasma relaxin levels were lower than in the mare that exhibited a normal pregnancy outcome after fluphenazine treatment, especially during the last 3 weeks of pregnancy.

FIG. 4 illustrates the mean daily plasma relaxin concentrations of gravid pony mares that grazed on endophyte-infected pasture. Data are shown relative to day of parturition. Systemic relaxin was consistently higher in the fluphenazine-treated (TRT) mares compared with the controls (CTL) during the last 15 days of gestation.

### DETAILED DESCRIPTION OF THE INVENTION

Relaxin is a placental hormone in horses that can be measured in systemic blood in high concentrations (Stewart, D. R. et al. 1982. Biol. Reproduct. 27:17-24; Stewart, D. R. et al. 1986. Endocrinology 119:1100-1104). Relaxin is a member of the family of insulin-like molecules that is found in highest tissue concentrations during pregnancy in many species including the rat, rabbit, pig and horse. This hormone has several important functions during pregnancy and at the time of parturition. For example, it promotes uterine growth to accommodate the growing fetus and inhibits myometrial contractions during pregnancy. In species other than horses, relaxin has been shown to important in maintaining pregnancy and in delivery (Sherwood, O. D. 1994. In: The Physiology of Reproduction, Second Edition. E. Knobil and J. D. Neill (eds.), Raven Press: New York, pp. 861-1009). In humans, it has been suggested that relaxin might be a useful epidemiological tool, as women with symptoms of impending miscarriage in early pregnancy had lower levels of relaxin compared to women with normal pregnancies (Stewart, D. R. et al. 1993. Clin. Endocrinol. 38:379-385).

The placenta is a primary source of relaxin in horses during pregnancy, although the ovary also contributes to relaxin production during the reproductive cycle. The purification of equine relaxin from placental tissue has led to its biochemical characterization (Stewart, D. R. and H. Papkoff. 1986. Endocrinology 119:1100-1104) and development of a homologous relaxin radioimmunoassay (Stewart, D. R. 1986. Endocrinology 119:1100-1104). While plasma relaxin levels are low in non-pregnant mares, during pregnancy relaxin levels increase from day 80, peak at day 175, and remain elevated until foaling (Stewart, D. R. et al. 1992. Biol. Reproduct. 46:648-652). However, shortly after expulsion of the placenta, relaxin titres decline rapidly and return to pre-gestational levels (Stewart, D. R. 1986. Endocrinology 119:1100-1104). Little information is available on the role of relaxin during pregnancy and parturition in the mare, although there are reports of compromised relaxin secretory patterns during at-risk pregnancies and complicated deliveries (Stewart, D. R. et al. 1992. Biol. Reproduct. 46:648-652).

It has now been found that relaxin is a biochemical marker of placental function and fetal well-being and can be used as a predictor of pregnancy outcome, to monitor placental disease progression, and to monitor treatment efficacy in mares with placental insufficiency.

A retrospective analysis of plasma relaxin was performed on mares (light breeds) with problematic pregnancies (Ryan, P. L. et al. 1997. Proc. 15^{th} Equine Nutrition and Physiology Symposium, Fort Worth, Tex., May 28-31). Factors contributing to problematic pregnancies included placentitis, premature placental separation, hydrops, oligohydrallantois, twinning, and mares with pituitary tumors. Blood was collected during the third trimester of pregnancy and plasma relaxin was determined using a homologous equine relaxin radioimmunoassay. In a population of mares with normal pregnancies and deliveries, systemic relaxin ranged from 45.0 to 85.0 ng/ml, with a mean weekly value of 63.0 ng/ml during the last seven weeks of gestation. Plasma relaxin declined markedly in mares with problematic pregnancies. Relaxin concentrations in a mare with placentitis declined during the last 45 days of gestation from 53.0 ng/ml (day 298 of gestation) to a low of 33.0 ng/ml (day 312 of gestation), with an average concentration of 46.0 ng/ml. Plasma relaxin was severely depressed in two mares with pituitary tumors as well, with mean levels of 14.1 and 8.6 ng/ml. Two mares with pregnancy-related fluid problems, one with hydrops and one with oligohydrallantois, had variable relaxin titres, with mean values of 33.2 and 42.3 ng/ml, respectively. An ultrasound scan of the pregnancy on day 302 of gestation indicated loss of fetal fluid with severe folding of the fetal membranes. The foal from the latter mare was euthanized at birth due to severe hypoxia and sepsis. In the case of a mare with a twin pregnancy, systemic relaxin declined from a high of 68.9 ng/ml on day 244 of gestation to a low of 6.1 ng/ml on day 301 of gestation. The mare delivered both foals, one of which was dead at birth and the second was euthanized at 4 weeks of age. In all pregnancies in which systemic relaxin was low, foals were born hypoxic and immature but survived with aggressive medical intervention. These data demonstrate the role of plasma relaxin as a biomarker of placental function in pregnant mares, with low levels of plasma relaxin a predictor of poor prognosis for the foal at birth.

In a second study, the effects of fescue toxicosis and fluphenazine on plasma relaxin concentrations in pregnant pony mares was determined. Fescue toxicosis is a condition that results from grazing on endophyte-infected fescue and results in placental insufficiency (altered placental function) in pregnant horses. Fluphenazine, a long-acting D₂-dopamine antagonist is a drug that is used to treat fescue toxicosis (Ryan, P. L. et al. 1998. Proc. 44^{th} Annual Convention of the American Association of Equine Practitioners 44:60-61). Mares grazed on endophyte-infected fescue pasture (80% infected) were treated with either saline or a one-time injection of 25 mg fluphenazine deconate (i.m.) on day 320 of gestation. Blood was collected daily from day 300 of gestation and assayed for relaxin levels. Mean relaxin concentrations in both groups of mares during the week prior to treatment were variable but not significantly different (saline, 53.3±8.5 ng/ml; fluphenazine, 62.0±9.9 ng/ml). In the two weeks prior to delivery, there was a marked difference in relaxin concentrations between the treatment groups, with the most dramatic effect seen during the last week of gestation (saline, 45.7±6.7 ng/ml; fluphenazine, 64.6±7.2 ng/ml). Three of the six saline-treated mares exhibited clinical signs of fescue toxicosis (placental thickening, stillbirth, and agalactia) while one fluphenazine-treated mare showed clinical signs of toxicosis. These data suggested that a one-time injection of fluphenazine increased circulating relaxin and improved pregnancy outcome by reducing the adverse effects of fescue toxicosis.

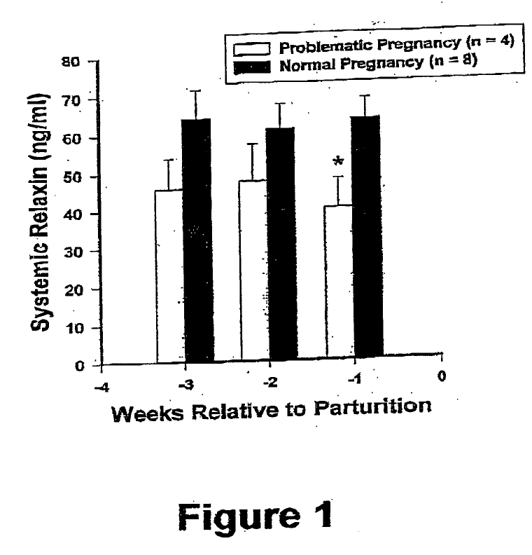

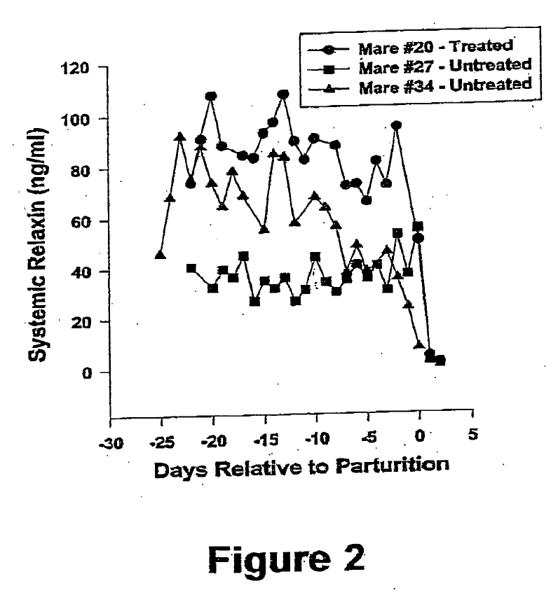

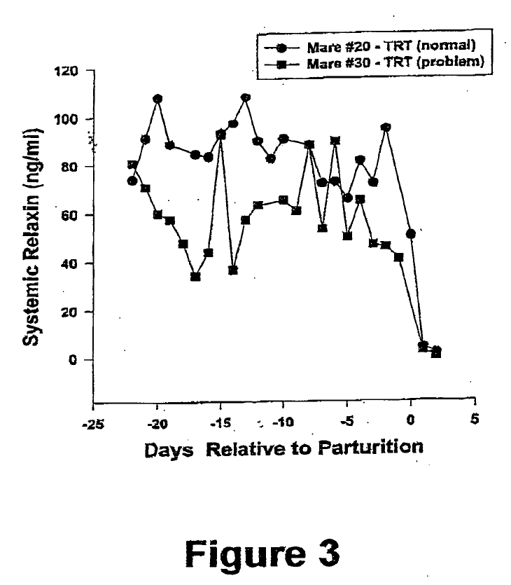

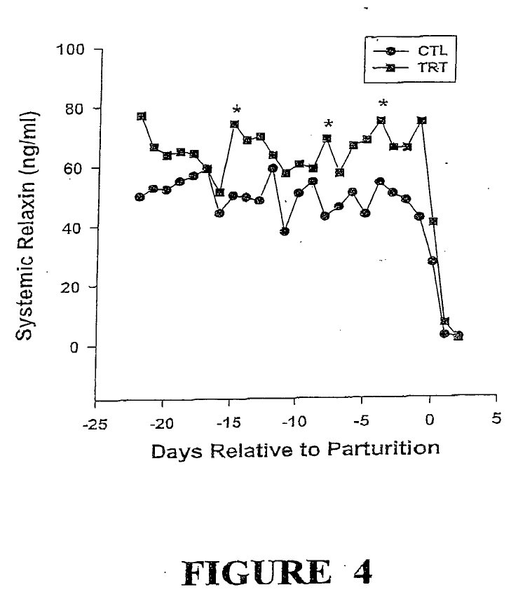

It has now been found that plasma relaxin levels are a useful predictor of treatment efficacy in pregnant mares with a disease or condition known to affect placental function. In the mares studied above, it has now been found that the improved pregnancy outcome was associated with an increase in circulating relaxin. Regardless of treatment, saline or fluphenazine, as a group, the mares with problematic pregnancies had lower systemic relaxin levels when compared to mares experiencing normal pregnancies (FIG. 1). Tracking of individual plasma relaxin levels in three mares, one treated with fluphenazine that experienced a normal pregnancy outcome (FIG. 2; mare #20) and two mares treated with saline that experienced problematic pregnancies (FIG. 2; mares #27 and #34) illustrates that plasma relaxin levels were consistently lower in the mares that later experienced a problematic pregnancy/delivery. FIG. 3 illustrates the predictive ability of plasma relaxin levels for a problematic pregnancy by comparing plasma relaxin profiles in two mares treated with fluphenazine. Although both of the mares had been treated with a drug to relieve the symptoms of fescue toxicosis, only one mare responded to treatment with a normal relaxin profile, i.e., higher levels of plasma relaxin. FIG. 4 also illustrates the mean daily plasma relaxin concentrations of gravid pony mares grazed on endophyte-infected pasture in relationship to the effect of fluphenazine treatment, relative to the day of parturition. The figure shows that systemic relaxin was consistently higher in the fluphenazine-treated (TRT) mares compared with the controls (CTL) during the last 15 days of gestation (*=values significantly different at p <0.05). These data demonstrate the ability of plasma relaxin levels as predictors of the efficacy of treatment outcome in horses affected by diseases or conditions that affect placental function.

The present invention is a method for predicting treatment efficacy in pregnant mares affected by a disease or condition that alters placental function and results in a problematic pregnancy or delivery in the mare. In the context of the present invention, “altered placental function” is a change in the function of the placenta that adversely affects the health or well-being of the fetus or delivered foal. In the context of the present invention “problematic pregnancy or delivery” is an adverse outcome to a pregnancy where the health of the fetus or foal is adversely affected such that the foal is born either dead or with life-threatening health problems that require immediate aggressive medical treatment. The method for predicting treatment efficacy relies on the measurement of plasma relaxin levels in the mares both during pregnancy and immediately before delivery whenever a therapeutic regimen (i.e., a drug or treatment) is administered to the mare to treat some disease or condition that produces an altered placental function. In the context of the present invention, “plasma relaxin” would include not only the parent compound known as equine relaxin but also other relaxin-like factors that are identified. The relaxin-like factors would include compounds with relaxin-like pharmacological activity and could be natural or synthetic compounds. A failure of plasma relaxin levels to increase with therapeutic intervention is predictive of a problematic pregnancy or delivery in the mare. One of skill would understand when relaxin levels would be useful for monitoring efficacy of treatments for altered placental function, treatments that would include but not be limited to systemic antibiotics, progestins, non-steroidal anti-inflammatory drugs and/or steroids, and conditions that produce altered placental function that would include but not be limited to placentitis, premature placental separation, placental thickening, and fescue toxicosis. The present invention is also a method to alleviate placental insufficiency by administering equine relaxin as a treatment. Finally, the method of the present invention can be adapted to the form of a diagnostic kit for predicting treatment efficacy or monitoring treatment efficacy in pregnant mares. The diagnostic kit would include a means of detecting equine relaxin in plasma both before and after treatment of the pregnant mare. A variety of means for detecting equine relaxin levels in plasma could be used by one of skill in developing such a diagnostic kit, means that would include but not be limited to radioimmunoassay.

The following non-limiting examples are provided to better illustrate the present invention.

### EXAMPLE

### Example 1

### Measurement of Plasma Relaxin Levels

An equine relaxin radioimmunoassay has been developed (Stewart, D. R. and H. Papkoff. 1986. Endocrinology 119:1100-1104) and was used to detect relaxin in bodily fluids. Briefly, purified equine relaxin standards or samples of unknowns were incubated with a rabbit anti-equine relaxin antiserum and radiolabeled equine relaxin (¹²⁵I-labeled relaxin). Twenty-four hours later, a secondary antibody, sheep anti-rabbit ε-globulin was added and seventy-two hours later the antigen-antibody complex was precipitated and counted. The concentration of equine relaxin present in the samples was determined by comparing results with a standard curve.

What is claimed is:

1. A method for predicting treatment efficacy in pregnant mares affected by a disease or condition that alters placental function and results in a problematic pregnancy or delivery in the mare comprising:
(a) measuring the levels of relaxin in plasma of a pregnant mare before administration of a drug or treatment;
(b) administering the drug or treatment to the mare; and
(c) measuring the levels of relaxin in plasma of the mare following administration of the drug or treatment from the first day of drug or treatment administration until the time of delivery in the mare, wherein a failure of the plasma relaxin levels to increase following drug or treatment administration is indicative of a problematic pregnancy or delivery in the mare.

2. A diagnostic kit for predicting treatment efficacy in preganant mares affected by a disease or condition that alters placental function and results in a problematic pregnancy or delivery in the mare comprising:
(a) a means for detecting equine relaxin levels in plasma of a pregnant mare before administration of a drug or treatment; and
(b) a means of detecting equine relaxin levels in plasma of a pregnant mare after administration of a drug or treatment from the first day of drug or treatment administration until the time of delivery in the mare.
